(19) United States
(12) Patent Application Publication
(10) Pub. No.: US 2025/0093344 A1
(43) Pub. Date: Mar. 20, 2025

(54) DIGITAL IMMUNOASSAY DETECTION AND ANALYSIS METHOD FOR PRECISE QUANTIFICATION OF PROTEINS

(71) Applicant: Bo YU, Panjin (CN)

(72) Inventors: Bo YU, Panjin (CN); Huan LI, Shanghai (CN)

(21) Appl. No.: 18/888,789

(22) Filed: Sep. 18, 2024

(30) Foreign Application Priority Data
Sep. 19, 2023 (CN) 202311209690.7

(51) Int. Cl. G01N 33/543; C12Q 1/28; G01N 21/64; G01N 33/58; G01N 33/68

(52) U.S. Cl. CPC G01N 33/54333; C12Q 1/28; G01N 21/6458; G01N 33/581; G01N 33/6803; G01N 2333/908

(57) ABSTRACT

A digital immunoassay detection and analysis method for precise quantification of proteins are provided. The digital immunoassay detection and analysis method for precise quantification of proteins includes the steps of: (s1) providing an aqueous phase mixture; (s2) mixing the aqueous phase mixture with a chromogenic substrate and an oil phase to form a first mixture; (s3) shearing of the first mixture to form a sheared water in oil second mixture; and (s4) measuring the first micro droplet to obtain the number of first droplets and obtain the quantitative detection result of protein. The second mixture contains a first micro droplet and a second micro droplet, The first micro droplet is a micro droplet containing a positive signal complex. The second micro droplet is a micro droplet containing a negative signal complex.

### TECHNICAL FIELD

The present invention patent belongs to the field of biological analysis. Specifically, it relates to a digital immunoassay detection and analysis method for precise quantification of proteins.

### BACKGROUND

For the determination of new biomarkers, enzyme-linked immunosorbent assay (ELISA) is no longer sufficient. For example, the sensitivity of this method is no longer sufficient for quantitative analysis of proteins such as P-tau-181, 217, 231 antigen and neurofibrillary light chain (NfL) associated with Alzheimer's disease. Although these have been included in clinical guidelines, traditional detection methods cannot achieve precise measurements due to the low levels of these substances in the blood. In addition, due to the large reaction system, the response of the reporter molecule is limited. The concentration of reporter molecule is too low, while the reaction system is too large, resulting in the effective signal concentration being diluted by the reaction solution.

In recent years, digital immunoassay method has received increasing attention due to its high sensitivity and is regarded as the new gold standard. For example, Quanterix's Simoa platform is a typical representative of the method. Firstly, the Simoa platform utilizes over 200,000 micron sized micropores as detection and sampling carriers, limiting the reaction system to the fL level, which is 1,000,000,000 times smaller in volume than the uL level of traditional ELISA methods. In this way, the signal generated by a reporter molecule is limited to a smaller system, and its concentration can reach the range that the instrument optical system can easily respond to (mg/mL level), and the number of observed molecules can be obtained by counting.

However, due to the inability of digital immune signals to sample all molecules and technical constraints or sample concentration issues, it cannot be guaranteed that all molecules can be sampled and detected. In other words, it cannot be guaranteed that every molecule can be ‘captured’ or ‘read’. In this case, the sampling results will be affected by randomness, which means that only some molecules can be sampled and detected, and there is a certain degree of uncertainty as to whether these molecules can represent the whole. At low concentrations, the sampling process follows Poisson Distribution, and a maximum of 200,000 micropores can only satisfy about 1-6000 digital immune signal conditions, with a number of digital immune signal wells greater than 99%. In addition, due to the presence of Poisson Distribution noise, the theoretical CV is ≥10% (or 20% if n is 25 or less), when the number of positive molecules (n) detected in the digital immunizations is less than 100. This determines that its linear range is narrow and can only be achieved in the state of 25-6000 molecules, which is difficult to meet the full digital requirements of practical applications.

In addition, the Simoa platform uses magnetic beads as capture units, and then introduces the magnetic beads into micropores for analysis. This process essentially involves secondary sampling, which undoubtedly increases the uncertainty of sampling. Due to the low efficiency of secondary sampling, according to relevant literature reports, the sampling efficiency of the Simoa platform HD-1 is only about 5%. This is mainly due to the limitation of the number of micropores by the chip area, as well as the efficiency issue caused by the introduction of magnetic beads into micropores.

Therefore, to improve the performance of digital immune detection, the key lies in increasing the number of micropores and improving the efficiency of secondary sampling. Although digital immune detection has enormous potential value, the high cost of the Simoa scheme makes it difficult to improve detection throughput and the narrow linear range makes it difficult to meet the needs of large-scale and low-cost applications.

At present, the method for digital detection by using micro droplets has been widely applied in digital PCR technology. The digital PCR systems on the market mainly use micro droplet generation technology, among which Bio-Rad's QX200 Droplet Digital PCR System is a typical representative. This system generates micro droplets through microfluidic chips or microfluidic valves, and each micro droplet can be seen as a micro reaction chamber that can perform independent PCR reactions. In Bio-Rad's system, the generation of micro droplets is achieved through a technique called “water-in-oil”, which involves adding surfactants to two immiscible liquids (such as water and oil) and forming micro droplets through physical shear or pressure driving. The size of each micro droplet is about 1 nanoliter, and each PCR reaction can generate approximately 20,000 micro droplets. These micro droplets are evenly distributed in the reaction tube during the PCR reaction, so that the number of DNA molecules in each micro droplet follows Poisson Distribution, thereby achieving digital PCR detection.

This technology has been applied in some fields. However, in contrast, digital immune detection based on micro droplets has not yet been commercially applied.

Therefore, it is of great significance to develop a highly sensitive, accurate, and efficient digital immunoassay detection method in this field.

### SUMMARY OF THE INVENTION

Provided is a highly sensitive, accurate, and efficient digital immunoassay detection method.

The first aspect of the present invention provides a digital immunoassay detection and analysis method for precise quantification of proteins, comprising the steps of:
(s1) providing an aqueous phase mixture comprising the following complexes:
   (A) a positive signal complex: a magnetic bead—antibody—antigen—detection antibody enzyme label, and/or a magnetic bead—antibody—antigen—detection antibody complex; and (B) a negative signal complex: a magnetic bead—antibody complex;
(s2) mixing the aqueous phase mixture with a chromogenic substrate and an oil phase to form a first mixture;
(s3) mechanically shearing of the first mixture to form a sheared water-in-oil second mixture;
wherein, the second mixture comprises a first micro droplet and a second micro droplet; the first micro droplet is a micro droplet containing a positive signal complex; the second micro droplet is a micro droplet containing a negative signal complex; and
(s4) measurement of the first micro droplet to obtain the number of the first micro droplets and obtain the quantitative detection result of the proteins.

In another preferred embodiment, the method further comprises the following sub steps in step (s1):
(s1a) providing an aqueous monodisperse magnetic bead solution;
(s1b) adding a certain proportion of capture antibodies (a first antibody) to the magnetic bead solution to couple the magnetic beads with the capture antibodies, thereby forming a magnetic bead—antibody complex, followed by washing;
(s1c) adding an antigen (a first antigen or protein to be tested) to a solution containing the magnetic bead—antibody complex and reacting to form a magnetic bead—antibody—antigen complex, followed by washing;
(s1d) adding a detection antibody containing biotin or horseradish peroxidase (HRP) (a second antibody) to a solution containing the magnetic bead—antibody—antigen complex and reacting to form a magnetic bead—antibody—antigen—detection antibody complex, followed by washing;
wherein, when a detection antibody containing biotin is added, proceed to step (s1e); when a detection antibody containing horseradish peroxidase is added, proceed directly to step (s2);
(s1e) adding an enzyme labeled molecule to a solution containing the magnetic bead—antibody—antigen—detection antibody complex to form a magnetic bead—antibody—antigen—detection antibody—enzyme label, followed by washing; and
(s1f) optionally, adding a fluorescent antibody against the capture antibody (a negative signal antibody) to a solution containing the magnetic bead—antibody—antigen—detection antibody—enzyme label, so that the magnetic bead is labeled with fluorescence (a negative signal), followed by washing.

In another preferred embodiment, the particle size of the magnetic bead is 0.03-100 μm.

In another preferred embodiment, the particle size of the magnetic bead is 1-5 μm.

In another preferred embodiment, the method further comprises the following sub steps in step (s3):
(s3a) mechanically shearing of the first mixture, wherein the shearing is dispersing the first mixture by using mechanical segmentation (high-speed homogenizer) to uniformly disperse magnetic beads into micro droplets, thereby forming a water-in-oil second mixture; and
(s3b) subjecting the second mixture to static reaction for a period of time.

In another preferred embodiment, the method further comprises the following steps between steps (s3) and (s4): washing the sheared water-in-oil second mixture to remove empty droplets that do not contain any first or second micro droplets.

In another preferred embodiment, the method further comprises the step in step (s4): measuring the second micro droplet to obtain the number of the second micro droplet and the ratio of the number of the first micro droplet to the second micro droplet, thereby obtaining a quantitative detection result of the proteins.

In another preferred embodiment, the method further comprises the following step in step (s2):
(s2a) mixing a solution containing the magnetic bead—antibody—antigen—detection antibody—enzyme label or magnetic bead—antibody—antigen—detection antibody complex with the chromogenic substrate evenly, and immediately adding the oil phase reagent to form the first mixture.

In another preferred embodiment, the oil phase is a fluorinated oil selected from the group consisting of: HFE7500, FC40, Fluo-Oil 7500, Fluo-Oil 40, Fluo-oil 135, Fluo-Oil 200, and a combination thereof.

In another preferred embodiment, a fluorinated surfactant needs to be added to the fluorinated oil to form the final oil phase.

In another preferred embodiment, the fluorinated surfactant is selected from the group consisting of: fluoroalkyl polyether sulfate, fluoroalkyl polyethylene glycol ether sulfate, fluorocarbon-based alcohol and fluorocarbon-based acid, PFPE-PEG polymer, PFPE-b-PPO-PEO-PPO-b-PFPE, and a combination thereof.

In another preferred embodiment, the enzyme labeled molecule is streptavidin-HRP.

In another preferred embodiment, the washing is carried out by adsorbing and intercepting micro droplets wrapped with individual magnetic beads through magnetic force, thereby washing and enriching the magnetic beads.

In another preferred embodiment, the magnetic bead is a magnetic bead with fluorescent properties or a magnetic bead without fluorescent properties.

In another preferred embodiment, the magnetic bead is a magnetic bead with fluorescent properties.

In another preferred embodiment, the method uses multi-color magnetic beads with fluorescent properties for quantitative detection of multiple substances.

In another preferred embodiment, wherein the method is used for measuring n proteins, and the value of n is 1-200, preferably 1-20, and more preferably 1-10.

In another preferred embodiment, when detecting n proteins, the second mixture contains the first micro droplets corresponding to each protein.

In another preferred embodiment, the total number of the first and second micro droplets in the second mixture effectively quantified by the method is greater than 50,000.

In another preferred embodiment, the total number of the first and second micro droplets in the second mixture effectively quantified by the method is ranging from 50,000 to 1 million.

In another preferred embodiment, the total number of the first and second micro droplets in the second mixture effectively quantified by the method increases with the expansion of the optical camera target surface.

In another preferred embodiment, the magnetic beads with fluorescent properties emit fluorescence at different wavelengths, comprising green (520 nm), yellow (490 nm), red (610 nm), blue (450 nm), as well as rare earth europium fluorescence and rare earth terbium fluorescence.

In another preferred embodiment, the magnetic beads further have different modifications.

In another preferred embodiment, the magnetic bead modification comprises carboxyl, amino, p-toluenesulfonic acid, NHS, Biotin, and streptavidin (SA).

In another preferred embodiment, the magnetic bead is a magnetic bead without fluorescent properties.

In another preferred embodiment, when the magnetic bead is a magnetic bead without fluorescent properties, step (s1f) is used to fluorescently label the magnetic beads.

In another preferred embodiment, the antibody is conjugated with carboxyl groups in step (s1b).

In another preferred embodiment, in step (s3), a second mixture containing more than 10,000 micro droplets is rapidly formed during mechanical shear treatment of the first mixture.

In another preferred embodiment, in step (s3), a second mixture containing 5-10 billion micro droplets is rapidly formed during mechanical shear treatment of the first mixture.

In another preferred embodiment, in step (s3), the volume of the water-in-oil second mixture is 10-1,000 uL.

In another preferred embodiment, the total volume of micro droplets in the second mixture is 10-1,000 uL.

In another preferred embodiment, the concentration of micro droplets in the second mixture is 0.5-90% (v/v).

In another preferred embodiment, the size of the micro droplet is 1-100 μm.

In another preferred embodiment, the mechanically shearing is carried out by homogenizing the first mixture using a high-speed homogenizer.

In another preferred embodiment, the speed of the high-speed homogenizer is ranging from 2,000 to 100,000 RPM.

In another preferred embodiment, the speed of the high-speed homogenizer is 10,000 RPM.

In another preferred embodiment, the ratio D1/D2 of the diameter D1 of the first micro droplet to the diameter D2 of the magnetic bead contained therein is 1.2-20, preferably 1.5-5, more preferably 1.6-4.

In another preferred embodiment, the ratio D1/D2 of the diameter D1 of the first micro droplet to the diameter D2 of the magnetic bead contained therein is 1.8-3.

In another preferred embodiment, the total content of the first and second micro droplets in the water-in-oil second mixture is less than 30%, preferably less than 20%, and more preferably less than 10%, relative to a total content of empty micro droplets in the second mixture.

In another preferred embodiment, the content of the first micro droplets in the water-in-oil second mixture is greater than 30%.

In another preferred embodiment, the chromogenic substrate comprises ADHP (OxiRed probe), BBTP, and FDG.

In another preferred embodiment, the chromogenic substrate is ADHP (OxiRed probe).

In another preferred embodiment, there is no cross interference between the spectrum of the substance produced by the enzymatic reaction between the positive complex and the chromogenic substrate and the emission wavelength of the negative complex.

In another preferred embodiment, in step (s3b), the conditions for the static reaction are: reacting at 37° C. for 5 minutes.

In another preferred embodiment, in step (s4), the fluorescence signals of the green and red channels are used to accurately count the number of the first and second micro droplets after the reaction.

In another preferred embodiment, in step (s4), a droplet imaging instrument is used for fluorescence imaging, which can capture the number of first micro droplets n and the number of second micro droplets m within one detection cycle.

In another preferred embodiment, in step (s4), the droplet imaging instrument uses a monochrome or color camera, which is a CCD camera or CMOS camera with more than 5 million pixels, or a monochrome or color camera.

In another preferred embodiment, in step (s4), a special low imaging and low distortion lens is selected to enable clear resolution of a magnetic bead.

In another preferred embodiment, in step (s4), the measurement comprises measuring the second micro droplet by using a fluorescent antibody to bind to a magnetic bead—antibody complex and labeling the magnetic bead with a fluorescent label (negative signal label); alternatively, magnetic beads with fluorescent properties can be selected, based on the characteristic of no cross interference between emission wavelength thereof and the spectrum of the substance produced by the enzymatic reaction in step (s3), in order to achieve the determination of the second micro droplet.

In another preferred embodiment, when the magnetic bead is a magnetic bead with fluorescent properties, the fluorescence of the magnetic bead can produce different fluorescence intensities depending on the content of fluorescent dyes, thereby further identifying and resolving the magnetic bead.

The second aspect of the present invention provides a digital immunoassay device for quantification of proteins, wherein the device comprises:
(a) a mixing module, which is configured to form an aqueous phase mixture comprising the following components:
   (A) a positive signal complex: a magnetic bead—antibody—antigen—detection antibody enzyme label, and/or magnetic bead—antibody—antigen—detection antibody complex; and (B) a negative signal complex: a magnetic bead—antibody complex;
(b) a shearing module, which is configured to add a chromogenic substrate and an oil phase to the aqueous phase mixture to form a first mixture; and perform a mechanical shearing to form a sheared water-in-oil second mixture, which contains first and second micro droplets; the first micro droplet is a micro droplet containing a positive signal complex; the second micro droplet is a micro droplet containing a negative signal complex; and
(c) a digital detection module, which is configured to measure the first micro droplet, thereby obtaining the number of the first micro droplet and obtaining the quantitative detection result of the proteins.

In another preferred embodiment, the particle size of the magnetic bead is 0.03-100 μm.

In another preferred embodiment, the particle size of the magnetic bead is 1-5 μm.

In another preferred embodiment, the magnetic bead is a magnetic bead with fluorescent properties or magnetic beads without fluorescent properties.

In another preferred embodiment, the oil phase is a fluorinated oils selected from the group consisting of: HFE7500, FC40, Fluo-Oil 7500, Fluo-Oil 40, Fluo-oil 135, Fluo-Oil 200, and a combination thereof.

In another preferred embodiment, a fluorinated surfactant needs to be added to the fluorinated oil to form the final oil phase.

In another preferred embodiment, the fluorinated surfactant is selected from the group consisting of: fluoroalkyl polyether sulfate, fluoroalkyl polyethylene glycol ether sulfate, fluorocarbon-based alcohol and fluorocarbon-based acid, PFPE-PEG polymer, PFPE-b-PPO-PEO-PPO-b-PFPE, and a combination thereof.

In another preferred embodiment, in step (s3), the volume of the water-in-oil second mixture is 10-1,000 uL.

In another preferred embodiment, the concentration of micro droplets in the second mixture is 0.5-90% (v/v).

In another preferred embodiment, the size of the micro droplets is 1-100 μm.

In another preferred embodiment, the mechanically shearing is carried out by homogenizing the first mixture using a high-speed homogenizer.

In another preferred embodiment, the speed of the high-speed homogenizer is ranging from 2,000 to 100,000 RPM.

In another preferred embodiment, the speed of the high-speed homogenizer is 10,000 RPM.

In another preferred embodiment, the ratio D1/D2 of the diameter D1 of the first micro droplet to the diameter D2 of the magnetic bead contained therein is 1.2-20, preferably 1.5-5, more preferably 1.6-4.

In another preferred embodiment, the ratio D1/D2 of the diameter D1 of the first micro droplet to the diameter D2 of the magnetic bead contained therein is 1.8-3.

In another preferred embodiment, a total content of the first and second micro droplets in the water-in-oil second mixture is less than 30%, preferably less than 20%, and more preferably less than 10%, relative to a total content of empty micro droplets in the second mixture.

In another preferred embodiment, the chromogenic substrate is ADHP (OxiRed probe).

In another preferred embodiment, in step (s4), a droplet imaging instrument is used for fluorescence imaging, which can capture the number of first micro droplets n and the number of second micro droplets m within one detection cycle.

In another preferred embodiment, the method is used to determine n proteins, and the value of n is 1-200, preferably 1-20, and more preferably 1-10.

In another preferred embodiment, when detecting n proteins, the second mixture contains the first micro droplets corresponding to each protein.

In another preferred embodiment, when detecting n proteins, the second mixture contains the first micro droplets corresponding to each protein.

In another preferred embodiment, the total number of the first and second micro droplets in the second mixture effectively quantified by the method is greater than 50,000.

In another preferred embodiment, the total number of the first and second micro droplets in the second mixture effectively quantified by the method is ranging from 50,000 to 1 million.

It should be understood that within the scope of the present invention, each technical feature of the present invention described above and in the following (such as examples) may be combined with each other to form a new or preferred technical solution, which is not listed here due to space limitations.

### BRIEF DESCRIPTION OF DRAWINGS

FIG. 1 shows the single component high-sensitivity approach of the digital immunoassay analysis method.

FIG. 2 shows the single-molecule detection analysis method with multiple proteins detected simultaneously.

FIG. 3 shows the single molecule detection and analysis method, which is a fast way.

FIGS. 4A-4B show: FIG. 4A after adding aqueous phase droplets to the oil phase, without using high-speed stirring or homogenization operation, the magnetic bead liquid phase floats in the upper layer, while the oil is in the lower layer; FIG. 4B after using a mechanical shearing to form immune micro droplets, it is in the state of an water-in-oil magnetic bead solution, where the oil phase is in the lower part and the droplets encapsulating the magnetic beads are in the upper part.

FIG. 5 shows the detection chip of the present invention, consisting of two layers of non-fluorescent materials with a 100 um interlayer in the middle (unit: mm).

FIGS. 6A-6C show: FIG. 6A fluorescence photos taken under 1× objective conditions, green channel fluorescence photos (water-in-oil fluorescence magnetic bead photos comprising negative and positive droplets); FIG. 6B fluorescence photos taken under 1× objective conditions, red channel fluorescence photos (positive droplets after HRP reaction); and FIG. 6C fluorescence photos taken under 1× objective conditions with the green and red channel fluorescence photos combined.

### DETAILED DESCRIPTION

Through extensive and deep research, the inventor has developed for the first time a highly sensitive, accurate, and efficient digital immunoassay method for precise quantification of proteins. Specifically, it comprises the following steps: (s1) providing an aqueous phase mixture comprising the following complexes: (A) a positive signal complex: a magnetic bead—antibody—antigen—detection antibody—enzyme label, and/or a magnetic bead—antibody—antigen—detection antibody complex; and (B) a negative signal complex: a magnetic bead—antibody complex; (s2) mixing the aqueous phase mixture with a chromogenic substrate and an oil phase to form a first mixture; (s3) mechanically shearing of the first mixture to form a sheared water-in-oil second mixture; wherein, the second mixture comprises a first micro droplet and a second micro droplet; the first micro droplet is a micro droplet containing a positive signal complex; the second micro droplet is a micro droplet containing a negative signal complex; and (s4) measuring the first micro droplet to obtain the number of the first micro droplets and obtain the quantitative detection result of the proteins. On this basis, the present invention has been completed.

### Terms

As used herein, the abbreviation “HRP” refers to horseradish peroxidase, which can bind with streptavidin to form a stable complex.

As used herein, the abbreviation “ADHP” is a highly sensitive and stable peroxidase substrate that selectively detects hydrogen peroxide (H₂O₂). In the presence of horseradish peroxidase (HRP) system, this substantially colorless and non-fluorescent ADHP reacts with H₂O₂ in a 1:1 chemical quantitative ratio to generate strong red fluorescence of Resorufin, which can be analyzed by fluorescence spectrophotometer and Microplate reader. Many enzyme reactions can produce H₂O₂, therefore ADHP can be used to detect the biological activity of many different enzymes in coupled enzymatic reactions.

As used herein, “microsphere” and “magnetic bead” can be used interchangeably, referring to a magnetic bead with or without fluorescent properties in the present invention.

As used herein, “the first antigen of the present invention”, “the antigen of the present invention”, and “the protein to be tested of the present invention” can be used interchangeably, referring to the protein to be accurately quantified.

As used herein, the term ‘comprising’ can be open, semi closed, or closed. In other words, the term includes the following limitations: “containing”, “substantially composed of”, “composed of”.

### ELISA

Enzyme linked immunosorbent assay (ELISA) is a commonly used protein quantitative analysis method. Since its introduction in the 1970s, this technology has been widely used in various scientific research and has become an indispensable classic technique in the field of biomedical research and development. ELISA is a basic immunoassay method that utilizes enzymatic reactions to quantitatively analyze the binding of antigens to antibodies, and it is used for the identification of peptides, proteins, antibodies, hormones, and so on. Over the past few decades, due to its high sensitivity and high-throughput, it has been the preferred technique for protein quantitative analysis and is widely recognized as the “gold standard” in the industry. The basic reaction process involves the specific binding of antibody-coated well plates with antigen in the sample, followed by the specific binding of detection antibodies labeled with reporter molecules to the antigens. Finally, the signals generated by the reporter molecules, such as color reactions, optical reactions, magnetic reactions, or electrochemical reactions, are used to indirectly quantify and analyze the antigens. Although this method has certain advantages, there are also some unavoidable drawbacks. The biggest issue is that it is essentially still a simulated quantitative testing technology, which can no longer meet the needs of measuring new biomarkers.

### Immune Reaction and Digital Immunoassay Detection Based on Micro Droplets

Unlike molecular hybridization reactions that only involve the interaction of two molecules (probe and target), immune reactions typically involve multi-faceted interactions of antigens, antibodies, and auxiliary molecules.

Therefore, in order to effectively carry out immune reactions, it is necessary to use carriers containing antibodies to capture and enrich target proteins. This method has been widely used in various immunoassay techniques, such as enzyme-linked immunosorbent assay (ELISA).

Secondly, regarding the requirement for signal amplification. In immune detection, due to the extremely small number of target molecules, additional reporter molecules need to be introduced to amplify the detection signal. This is usually achieved by introducing fluorescent or enzyme labeled reporter molecules, which can bind to target molecules or antibodies to generate detectable signals.

Finally, it is necessary to consider the characteristics of Poisson Distribution. In droplet microfluidic systems, each droplet can be considered as an independent reaction chamber, therefore, the number of molecules in each droplet should follow Poisson Distribution.

In digital immunoassay detection of droplets, the Poisson Distribution formula can be used to describe the distribution of target molecules in droplets. The Poisson Distribution formula is as follows:

$$P ( x = k ) = \frac{{λ}^{k}}{k !} {e}^{- λ} ( k = 0 , 1 , 2 … ) ;$$

wherein, P(x) is the probability of containing x target molecules in the droplet, and λ is the average number of target molecules. According to the characteristics of Poisson Distribution, as the average frequency (λ) of an event increases, the probability (P) of the event also increases accordingly.

However, it is necessary to control the number of target molecules containing two or more molecules in the droplet, that is, the number when k>1, such as λ=0.1, k=1, P₁≈0.090, the cumulative probability of k>1 P_{1-∞}≈0.0046, P₁/P_{1-∞}≈20, that is, when λ=0.1, the ratio of the number of droplets containing only one molecule to the number of droplets containing two or more molecules is 20, and the probability of droplets with K=0 is 0.90, which can be understood as 90% of droplets being empty droplets, 9% of droplets containing only one molecule, and 0.47% of droplets containing two or more molecules. The present invention sets the above conditions as the minimum λ standard. The presence of empty droplets greatly reduces the number of effective droplets containing magnetic beads that can be observed in the end. To improve the efficiency of observing magnetic beads, effective measures should be taken to eliminate empty droplets.

λ is the average number of target molecules, which is the ratio of the number of detected molecules (n) to the detection unit (m). Therefore, when λ≤0.1, effectively increasing the number of m is the key technology for achieving digital detection.

In addition to the characteristics of Poisson Distribution, digital immunoassay detection of droplets also needs to consider other factors, such as antibody capture and enrichment, signal amplification, etc.

By comprehensively considering these factors, the inventor has designed an efficient digital immunoassay detection method for droplets, thereby achieving sensitive detection of target molecules.

These are the key factors that need to be considered in droplet based digital immunoassay detection, and only by meeting these conditions can effective digital immunoassay detection be achieved.

### Digital Immunoassay Detection and Analysis Method for Precise Quantification of Proteins

The present invention utilizes micro droplet technology to disperse magnetic beads into water-in-oil micro droplets within 1-10 seconds through mechanical emulsification dispersion method. Magnetic beads will be randomly encapsulated in micro droplets to form water-in-oil droplets. The number of magnetic beads in the micro droplets follows Poisson Distribution, and then magnetic force is used to wash and trap the micro droplets containing magnetic beads, separating the empty droplets and significantly increasing the number of micro droplets containing magnetic beads that can be observed.

The present invention chooses to use monodisperse magnetic beads with a particle size of 1-5 μm. These magnetic beads can be widely used in the field of life sciences, including products from companies such as Dynabeads, MagSi, MyOne, M-270, Dynal Biotech, Sera-Mag, etc. The particle size range of these magnetic beads is from 0.03 μm to 100 μm. The magnetic beads used in the present invention have or do not have fluorescent properties, and can emit light of different wavelengths such as green (520 nm), yellow (490 nm), red (610 nm), blue (450 nm), as well as rare earth europium fluorescence and rare earth terbium fluorescence. These magnetic beads also have different modifications, including carboxyl, amino, p-toluenesulfonic acid, NHS, Biotin, and streptavidin (SA).

The present invention uses fluorinated oil as the oil phase, which is widely used in the field of life sciences. Common fluorinated oils include HFE7500, FC40, Fluo Oil 7500, Fluo Oil 40, Fluo Oil 135, and Fluo Oil 200. Adding fluorinated surfactants to fluorinated oil can form an oil phase. Common surfactants include fluoroalkyl polyether sulfate, fluoroalkyl polyethylene glycol ether sulfate, fluorocarbon-based alcohol and fluorocarbon-based acid, PFPE-PEG polymer, PFPE-b-PPO-PEO-PPO-b-PFPEs, etc. After mixing the above oil phase with aqueous phase, water-in-oil micro droplets can be rapidly formed using a high-speed homogenizer.

The aqueous phase of the present invention is a magnetic bead solution. By adding the magnetic bead solution to the oil phase solution and using a high-speed homogenizer, the magnetic beads can be dispersed into monodisperse water-in-oil droplets, with a controlled number of 500,000 magnetic beads. Using 100 μL of aqueous phase, it is estimated that millions to billions of micro droplets can be generated each time. Under these conditions, the magnetic beads are present in the form of a single bead encapsulated within a single microdroplet. Due to the excellent properties of surfactants in water-in-fluorinated oil, micro droplets can be present stably in the oil phase without leaking fluorescent substances to each other.

The water-in-oil magnetic beads can temporarily and stably adsorb onto the glass slide after being attracted by a magnet. The remaining droplets that do not contain magnetic beads are washed away by washing with the oil phase. At this time, the water-in-oil magnetic beads form a monodisperse magnetic bead layer on the glass slide, which can be observed by fluorescence imaging. A single magnetic bead can be clearly distinguished.

The present invention selects fluorescence microscope lenses with low deformation and low dispersion, with priority given to 1×, 2×, 4×, 10×, 20×, 40×, 60×, and 100× objectives. By adjusting the optical configuration, ensure that the formed phase field can observe the fluorescence signals generated by the magnetic beads and substrate. The present invention uses a dual channel fluorescent filter, which includes at least two channels selected from the group consist of: a DAPI channel with an emission wavelength of about 450-500 nm; a FITC channel with an emission wavelength of approximately 515-530 nm; a GFP channel with an emission wavelength of approximately 509-525 nm; Alexa Fluor 488 channel with an emission wavelength of approximately 519-545 nm; Cy3 channel with an emission wavelength of approximately 565-615 nm; Rhodamine Red-X channel with an emission wavelength of approximately 600-680 nm; Texas Red channel with an emission wavelength of approximately 600-680 nm; Cy5 channel with an emission wavelength of approximately 665-725 nm. The present invention uses a CCD or CMOS camera to capture fluorescent images, with the target size of the selected camera matching the lens. For example, cameras in sizes such as 4/3 inches, 1.2 inches, 1.1 inches, 1 inch, 1/1.1 inches, 1/1.2 inches, ⅔ inches, 1/1.8 inches, ½ inches, ⅓ inches, 36/24 millimeters, 45/60 millimeters, and 60/60 millimeters can be used. The pixel count of a camera can range from 1 million to 100 million pixels. The pixel size should match the numerical aperture (NA) of the selected objective lens. For example, when using a 1× objective lens with an NA of 0.045, according to the Rayleigh criterion (0.61λ/NA), its resolution at a wavelength of 500 nm is approximately 4.35 um. Therefore, the present invention can choose a pixel size of 3.45 um (⅔ inch camera) that matches it. In the case of 2-5 um magnetic beads, a pixel bright spot on the phase field can represent a magnetic bead signal.

After the immune reaction, mix the magnetic beads with the enzyme substrate at low temperature (2-8° C.) evenly, then immediately add the oil phase reagent and use a high-speed homogenizer for 1s-60s dispersion. This can evenly disperse the magnetic beads into the micro droplets. Due to the fact that the number of empty droplets is much larger than the number of magnetic beads, according to the Poisson Distribution principle, magnetic beads will be individually and uniformly dispersed into a single droplet. The size of magnetic bead droplets will be larger than the diameter of the magnetic beads, and the measured droplet size is between 1-10 times the size of the magnetic beads. The size of droplets is mainly affected by the speed of the high-speed homogenizer, the content of oil phase surfactants, and the action time of the high-speed homogenize. The typical droplet size is between 1-2 times the size of magnetic beads. Due to the shear force, the droplet size follows a normal distribution. In order to control the uniform distribution of droplets as much as possible, the reaction system parameters can be adjusted, including the speed of high-speed homogenizer, the content of oil phase surfactants, and the length of the high-speed homogenizer action time. During the color development process after the reaction, enzyme fluorescent substrates are used for color development. Commonly used substrates include ADHP (OxiRed probe) for horseradish peroxidase, BBTP for alkaline phosphatase, and FDG substrate for galactosidase.

The final resulting first microdroplets are characterized by containing both fluorescent magnetic beads and fluorescent substrate coloration, and they satisfy the following conditions simultaneously: 1) droplets containing only fluorescent magnetic beads; 2) droplets containing fluorescent magnetic beads and also exhibiting fluorescence from enzymatic substrate coloration; 3) excluding microdroplets that solely exhibit fluorescence from substrate coloration without containing fluorescent magnetic beads. All three conditions must coexist.

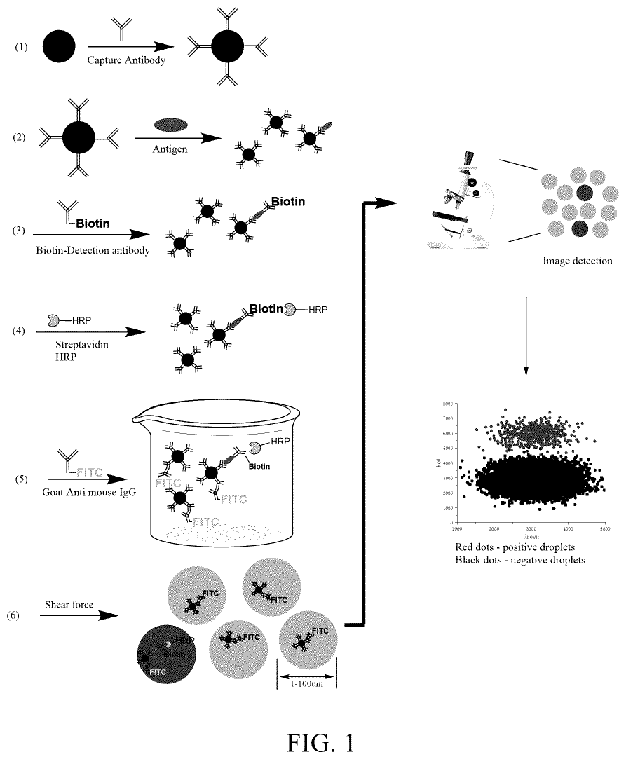

Magnetic beads are coupled with a certain proportion of capture antibodies, and after washing the beads, they are blocked with bovine serum albumin (BSA) solution. Followed by another wash, the resulting magnetic bead—antibody complexes are prepared for subsequent use. The concentration of these magnetic bead—antibody complexes is determined by counting, confirming that there are 500,000 beads. With the number of magnetic beads controlled, the reaction system is controlled to 50 μL. The above magnetic beads are allowed to react with the antigen for 15 minutes, then washed three times with a magnetic frame PBST 200 μL. Afterward, biotinylated detection antibody solution is added, and the mixture is allowed to react for 15 minutes, then washed three times with a magnetic frame PBST 200 μL. Afterward, streptavidin-HRP is added, and the mixture is allowed to react for 15 minutes, then washed three times with a magnetic frame PBST 200 μL. Afterward, 10 μL of anti-mouse IgG-FITC is added, and the mixture is agitated for 10 minutes, then washed three more times with a magnetic frame PBST 200 μL. The above magnetic beads are configured into a 100 uL solution (50 uL pre-cooled PBS with 50 uL pre-cooled ADHP) using pre-cooled PBS (2-8° C.) and the reaction is shown in FIG. 1

After the above magnetic bead reaction solution is fully prepared, the liquid mixture is injected quickly into 100 L of oil for droplet generation at once, and dispersed using a high-speed homogenizer for 1-60s to form a water-in-oil droplet mixture. And the droplet mixture is reacted at 37° C. for 5 minutes, and the above droplets are washed with a magnetic frame to remove unreacted empty droplets.

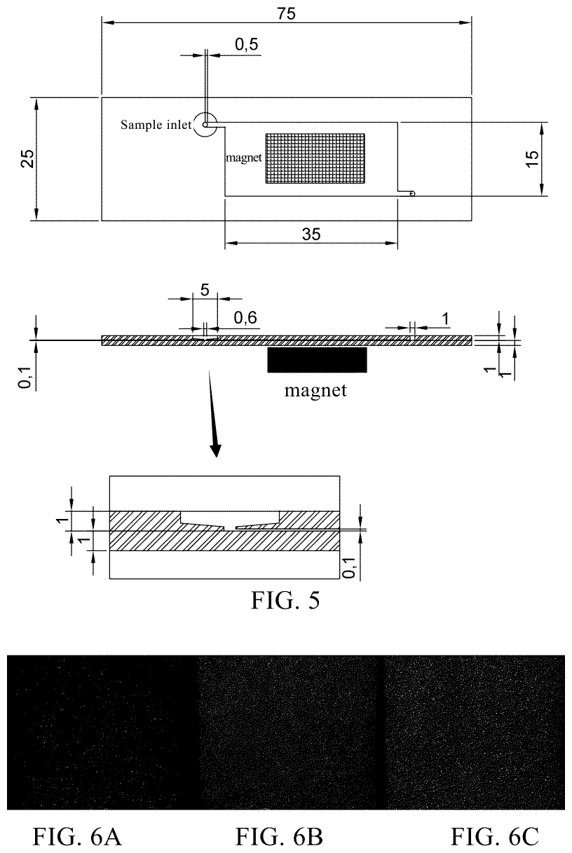

The above micro droplet solution is added to the detection cell for counting, and all green channel highlight counts n, and all red channel highlight counts m are recorded. The static detection cell consists of a fluorescence counting plate with a 100 μm sandwich in the center. The green and red channels, with wavelengths of 570 excitation, 585 nm emission and 470 excitation and 490 emission, are detected without interference. The detection method is shown in FIG. 1. The detection chip connected with this technique is shown in FIG. 5. After the solution is added, the magnetic beads will form a monodisperse bead layer on the surface corresponding to the 8*6 mm magnet due to magnetic force. The number of magnetic beads as well as the number of signal droplets are calculated by adjusting the algorithm threshold under ⅔ inch CMOS photography.

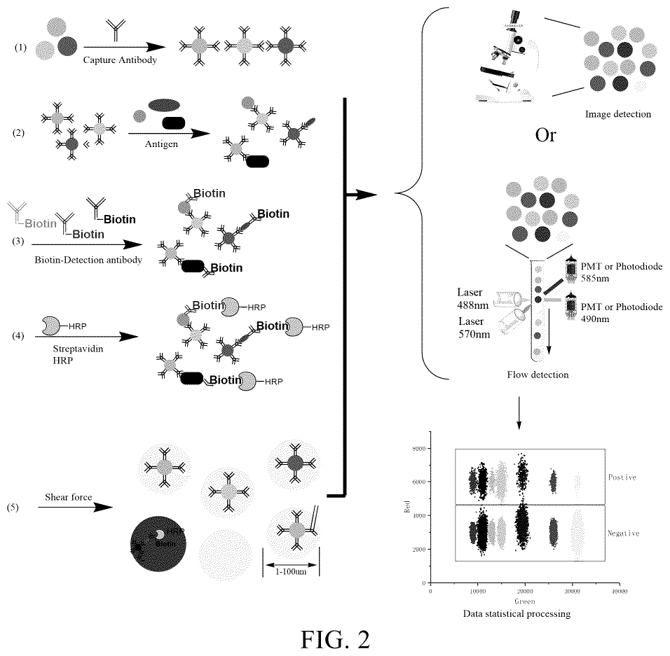

Multi colored fluorescent microspheres can also be used for multi marker detection. To achieve this, the instrument settings are adjusted, one color and multiple fluorescent microspheres with different intensities are employed as carriers, and each color and intensity of magnetic beads are coupled with a certain proportion of different capture antibodies. After cleaning the magnetic beads, they are washed and blocked with bovine serum albumin solution. After washing, magnetic bead—antibody complexes with different fluorescence intensities will be formed for subsequent use. The formed antibody coated magnetic beads are counted, and the concentration of magnetic beads is determined to be greater than 50,000, and then the magnetic beads are mixed. The total volume of magnetic beads is controlled and the reaction system is controlled to 50 uL, the above magnetic beads are reacted with antigens for 15 minutes, then washed three times with 200 uL of PBST using a magnetic frame. Afterward, biotinylated detection antibody solution is added, and the mixture is allowed to react for 15 minutes, then washed three times with 200 μL of PBST using a magnetic frame. Afterwards, streptavidin-HRP is added, and the mixture is allowed to react for 15 minutes, then washed three times with 200 L of PBST using a magnetic frame. The above magnetic beads are configured into a 100 uL solution (50 uL pre-cooled PBS with 50 uL pre-cooled ADHP) using pre-cooled PBS (2-8° C.) and the reaction is shown in FIG. 2.

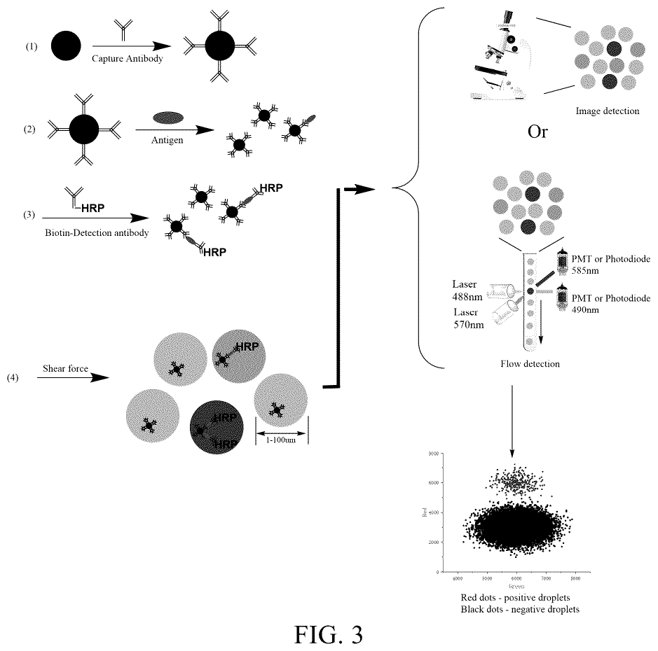

A fluorescent microsphere can also be used for rapid biomarker detection. To achieve this, the instrument settings are adjusted, a colored fluorescent microsphere is employed as a carrier, magnetic beads are coupled with a certain proportion of different capture antibodies. After cleaning the magnetic beads, they are washed, blocked with bovine serum albumin solution, and magnetic bead—antibody complexes are formed for subsequent use. The antibody-coated magnetic beads are counted to determine the concentration of magnetic beads to be greater than 50,000 beads and then the magnetic beads are mixed. The total volume of magnetic beads was controlled to 50 uL, the above magnetic beads are reacted with the antigen for 5 min, then washed three times with 200 uL of PBST using a magnetic frame. Followed by the addition of HRP detection antibody solution, the magnetic beads are allowed to react for 5 min, then washed three times with 200 uL of PBST using a magnetic frame. And the above beads were configured into a 100 uL solution using pre-cooled PBS (2-8° C.) (50 uL pre-cooled PBS with 50 uL pre-cooled ADHP) and the reaction is shown in FIG. 3.

The process of the micro droplet magnetic bead algorithm of the present invention is as follows:
1) reading a grayscale image;
2) applying adaptive thresholding to segment images into bright spots and backgrounds;
3) finding the connected components and separating the bright spots into different regions;
4) calculating the number of connected components (minus background).

The main advantages of the present invention include:
(a) By using a multi-channel micro droplet generation chip to construct a digital immunoassay detection system, it is possible to simply perform digital immunoassay detection on the micro droplets generated by the droplet generation device through the previous magnetic bead immune reaction, greatly improving sensitivity and achieving a detection capability of up to 1fg/mL.
(b) HRP enzyme is used in the present invention to amplify digital immune signals. At the same time, galactosidase, alkaline phosphatase, metal catalysis, and fluorescent particles can also be used to greatly expand the detection range and reduce the system's adaptability.
(c) The use of high-speed homogenization method to generate micro droplet magnetic bead complexes can quickly produce micro droplets in the order of billions of picoliters. At the same time, due to the use of magnetic bead separation technology, micro droplets containing magnetic beads can be effectively enriched, while empty droplets without magnetic beads can be effectively separated, resulting in extremely high sampling efficiency and effectively avoiding the waste of secondary sampling, greatly improving detection efficiency.
(d) The micro droplet fluorescence used can generate two different wavelengths of fluorescence, one channel is used to count the number of negative magnetic beads, and the other channel is used to count the number of positive magnetic beads. The concentration of the solution can be calculated through Poisson Distribution statistics.
(e) The present invention allows the use of colored magnetic beads to label different kinds of antibodies onto the magnetic beads, which in turn can provide multi-detection capability by utilizing the mega-generation capability of microdroplets. For example, a series of colored microspheres with different color ratios formed by doping different dyes in different proportions can be used as an analytical carrier, which in turn can achieve a theoretical 100 types of multi-component ultra-high sensitivity analytical capability.
(f) Due to the utilization of picoliter-scale micro droplets in the present invention, coupled with a short incubation process of just 5 minutes, rapid detection becomes feasible. The sensitivity calculation method is converted from digital immunoassay Poisson Distribution counting method to micro droplet fluorescence intensity based method, thereby achieving rapid detection and the ability to quantify proteins within 15 minutes.
(f) Due to the employment of picoliter-scale micro droplets and fluorescent magnetic beads in the present invention, a rapid detection process is achieved through a short incubation period such as 5 minutes, enabling the quantification of proteins within 15 minutes.
(g) By using digital immunoassay technology, the measurement range of droplet quantity can reach more than 4 log ranges, which is on the order of 100,000 to 1 million. Meanwhile, with the expansion of the target area of optical cameras, there is still potential for further improvement in droplet recognition capability. For example, using a 60*60 mm CMOS camera, such as the GSENSE6060 CMOS, and equipped with a 1× lens, can achieve droplet recognition capabilities in the billions on the target surface.

The present invention will be further illustrated below with reference to the specific examples. It should be understood that these examples are only for illustrating the present invention and not intend to limit the scope of the present invention. Experimental methods not specified in the following examples are usually under conventional conditions, such as conditions described in Sambrook et al., Molecular Cloning: Laboratory Manual (New York: Cold Spring Harbor Laboratory Press, 1989), or as recommended by the manufacturer. Unless otherwise specified, percentages and portions are weight percentages and weight portions.

### Example 1

### Highly Sensitive Digital Immunoassay Analysis of IL-2

### 1. Experimental Materials

The experimental materials used in this example are shown in Table 1.

TABLE 1

| Reagent name | Reagent components |
|---|---|
| Magnetic bead | PBS |
| washing solution | |
| CB buffer solution | 50 mM CB buffer solution (pH 9.5) |
| 3M ammonium | 3M ammonium sulfate solution (pH 9.5) |
| sulfate | |
| Wash buffer before | 20 mM PB PH 7.4 + 0.1% BSA + 0.9% Nacl + 0.05% Proclin300 |
| magnetic bead | |
| blocking | |
| Blocking buffer | 50 mM Tris-HCl PH 7.4 + 0.5% BSA + 0.9% Nacl + |
| | 0.05% Proclin300 |
| Magnetic bead | 50 mM Tris HCl pH 7.4 + 0.5% BSA + 1.0% gelatin + 0.9% NaCl + |
| working solution | 0.05% Proclin300 |
| Magnetic beads | MS300 Tosylactivated |
| Raw material | capture antibody for IL-2, biotinylated detection antibody, |
| | streptavidin HRP, IL2 antigen, anti-mouse IgG FITC |

### 2. Coating Method

(1) A 2 mL centrifuge tube was taken, 30 uL of sulfonated magnetic beads (MS300) was added, and the magnetic beads were washed three times with 0.3 mL of 20 mM PBS, and the washing solution was drained off on a magnetic frame afterward.
(2) The required raw materials, CB buffer solution, and 3M ammonium sulfate were added in proportion, as shown in Table 2.

TABLE 2

| | | |
|---|---|---|
| Ingredients/Magnetic Beads | 1 | mg |
| Coating antibody raw materials + CB | 200 | uL |
| buffer solution | | |
| 3M ammonium sulfate | 100 | uL |
| Total volume | 300 | uL |

(3) The centrifuge tube in step 2 was placed on a multi-purpose rotating shaker and reacted at 2-8° C. for 20 hours.
(4) The centrifuge tube in step 3 was placed on a magnetic frame for 5 minutes, the supernatant was removed, 0.3 mL of wash buffer before magnetic bead blocking was added, and washed three times.
(5) 0.3 mL of blocking buffer was added to the washed magnetic beads from step 4, mixed well, and reacted on a multi-purpose rotary shaker at 37° C. for 6 hours.
(6) step 4 was repeated.
(7) The magnetic beads were resuspended, 0.3 mL of magnetic bead working solution was added to each tube, and it was placed on a multi-purpose rotating shaker at 37° C. for 20 hours of reaction.
(8) The centrifuge tube in step 3 was placed on a magnetic frame for 5 minutes, the supernatant was removed, 0.3 mL of wash buffer before magnetic bead blocking was added, washed three times for 5 minutes each time.
(9) The washed magnetic beads were added to the magnetic bead working solution to prepare a 120 ug/mL magnetic bead working solution, and it was stored at 2-8° C.
(10) The solution concentration was configured to 10 million magnetic beads/mL.

### 3. Detecting Method

(1) A series of 50 uL sample solutions were taken, including 0, 1, 5, 10, 100, 400, 1000fg/mL. 10 calibrators of 5fg/mL and 10 calibrators of 100fg/mL each, 13 serum samples from healthy individuals and 10 serum samples from gastric cancer patients.
(2) 50 uL of magnetic beads was mixed with 50 uL of sample and reacted at 37° C. for 15 minutes. Afterwards, the magnetic beads were washed three times with 200 uL of PBST using a magnetic frame.
(3) 100 uL biotin coated detection antibody (0.1 ug) was added and reacted at 37° C. for 15 minutes. Afterwards, the magnetic beads were washed three times with 200 uL of PBST using a magnetic frame.
(4) 100 uL of HRP labeled with streptavidin (1:5000 fold dilution) was added and reacted at 37° C. for 15 minutes. Afterwards, the magnetic beads were washed three times with 200 uL of PBST using a magnetic frame.
(5) 100 uL of anti-mouse IgG FITC (0.1 ug) was added and reacted at 37° C. for 15 minutes. Afterwards, the magnetic beads were washed three times with 200 uL of PBST using a magnetic frame.
(6) After the above reaction was completed, the magnetic beads were dispersed into a PBS solution of 50 uL and pre cooled.

### 4. Droplet Generation

(1) 50 uL of pre cooled ADHP fluorescence generating substrate was added immediately, mixed well, and oil for droplet generation 200 uL was added immediately.
(2) The head of the high-speed homogenizer was inserted into the above mixture, the speed was adjusted to high-speed mode (10,000 RPM), and homogenized for 10s.

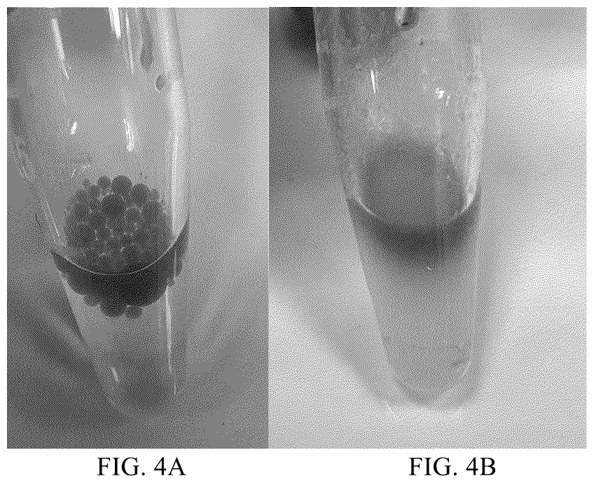

The stratification of the mixed solution and the resulting homogenate are shown in FIGS. 4A-4B.
(3) The above water-in-oil mixture was placed at 37° C. to react for 5 minutes.

Here, if the reaction is carried out first before mechanically hearing, it will cause an increase in the fluorescence of the empty droplets, which is not effective.

### 5. Detection

(1) The above reaction solution was placed on a magnetic frame, and it was attractively washed three times with oil for droplet generation, and finally it was resuspended in 20 uL of oil for droplet generation.
(2) The above-mentioned magnetic bead droplet mixture was added to the detection chip (FIG. 5) to form a single-layer distribution, and it was placed under a microscope for observation and detection.
(3) Photos of the green and red channels were taken, as shown in FIGS. 6A-6C.
(4) The fluorescence images under two excitation conditions: excitation at 570 nm and emission at 585 nm, and excitation at 470 nm and emission at 490 nm were analyzed. And the number of fluorescent droplets or magnetic beads under the two channels was counted. Droplets that did not contain magnetic beads were blank droplets with no fluorescence.
(5) The number of positive droplets containing magnetic beads and negative droplets containing magnetic beads were counted. The corresponding detection data list was shown in Table 3 below:

TABLE 3
Standard Curve Data for IL-2 in Digital Immunoassay

| Concentration of | Percentage of positive | Number of positive |
|---|---|---|
| calibrator fg/mL | magnetic beads | magnetic beads |
| 0 | 0.00100% | 4 |
| 1 | 0.01% | 45 |
| 5 | 0.05% | 214 |
| 10 | 0.10% | 440 |
| 100 | 1.04% | 4564 |
| 400 | 4.00% | 18532 |
| 1,000 | 9.63% | 40761 |

Regression analysis between the concentration of the calibration and the percentage of positive magnetic beads in Table 3 showed a linear relationship within the range of 1-1000 fg/mL of calibration concentration, which had a good correlation with the calculated values based on Poisson's theory.
(6) Each of the two IL2 standard solution concentrations, 5fg/mL and 100fg/mL, was measured 10 times independently using the above method. Calculated CV (repeatability data of digital immunoassay for IL2) as shown in Tables 4-5:

TABLE 4

| | Percentage of | Number of |
|---|---|---|
| | positive magnetic | positive magnetic |
| Test 5 fg/mL | beads | beads |
| 1 | 0.0500% | 213 |
| 2 | 0.0544% | 218 |
| 3 | 0.0459% | 225 |
| 4 | 0.0478% | 226 |
| 5 | 0.0478% | 202 |
| 6 | 0.0513% | 238 |
| 7 | 0.0532% | 247 |
| 8 | 0.0526% | 224 |
| 9 | 0.0498% | 201 |
| 10 | 0.0420% | 194 |

| | |
|---|---|
| SD | 0.00005135 |
| CV | 7.54% |

TABLE 5

| | Percentage of | |
|---|---|---|
| | positive magnetic | Number of positive |
| Test 100 fg/mL | beads | magnetic beads |
| 1 | 0.99978% | 4439 |
| 2 | 0.99997% | 4648 |
| 3 | 0.99985% | 4437 |
| 4 | 0.99988% | 4025 |
| 5 | 0.99980% | 4546 |
| 6 | 0.99993% | 4684 |
| 7 | 0.99981% | 4826 |
| 8 | 0.99997% | 4225 |
| 9 | 0.99989% | 4454 |
| 10 | 0.99982% | 4909 |

| | |
|---|---|
| SD | 0.0000006899 |
| CV | 0.01% |

Tables 4-5 shown that the CV of the 5fg/mL sample measured was 7.54%, and the CV of 100fg/mL was 0.01%.
(7) The above method was used to measure a group of 10 healthy samples and 13 corresponding serum samples from patients, and the result as shown in Table 6: Table 6 IL2 measurement data for healthy individuals and patients

TABLE 6
IL2 measurement data for healthy individuals and patients

| | Percentage of | Number of | |
|---|---|---|---|
| | positive | positive | |
| | magnetic | magnetic | Concentration |
| | beads | beads | pg/mL |
| Healthy | | | |
| individuals | | | |
| 1 | 0.3433% | 1452 | 34.3 |
| 2 | 0.2485% | 1234 | 24.9 |
| 3 | 0.2038% | 989 | 20.4 |
| 4 | 0.1123% | 517 | 11.2 |
| 5 | 0.4550% | 1832 | 45.5 |
| 6 | 0.3130% | 1462 | 31.3 |
| 7 | 0.3622% | 1714 | 36.2 |
| 8 | 0.1311% | 553 | 13.1 |
| 9 | 0.2668% | 1168 | 26.7 |
| 10 | 0.4002% | 1718 | 40.0 |
| Patients | | | |
| 1 | 1.8820% | 8184 | 188.2 |
| 2 | 2.1411% | 9652 | 214.1 |
| 3 | 2.0088% | 9178 | 200.9 |
| 4 | 2.4571% | 11956 | 245.7 |
| 5 | 1.7159% | 7976 | 171.6 |
| 6 | 2.1234% | 9377 | 212.3 |
| 7 | 2.4817% | 10070 | 248.2 |
| 8 | 1.8747% | 8043 | 187.5 |
| 9 | 1.9314% | 8226 | 193.1 |
| 10 | 2.4813% | 11714 | 248.1 |
| 11 | 1.6135% | 7833 | 161.4 |
| 12 | 2.2701% | 9227 | 227.0 |
| 13 | 1.8008% | 8961 | 180.1 |

The results in Table 6 indicated that the method of the present invention can effectively detect IL6 in serum.

### Example 2

### Digital Immune Sensitivity Analysis of Multiple Cytokines (IL-2, IL-5, IL-7, IL-8, IL-9, IL-12, IL-15)—Human Adaptive Immunity 7-Fold Quantitative Analysis

### 1. Experimental Materials

The experimental materials for this example are shown in Table 7

TABLE 7

| Reagent name | Reagent components |
|---|---|
| Activation | pH 5.0 MES |
| buffer solution | |
| / | 100 mg/mL EDC, prepared using pre cooled C-Buffer A and |
| | prepared for immediate use |
| / | 100 mg/mL NHC, prepared with pre cooled C-Buffer A, ready |
| | for immediate use |
| Binding buffer | 0.02M pH 8.0 boric acid buffer solution |
| Blocking buffer | 50 mM pH 7.4 Tris + 0.1% Tween-20 + 1% BSA + 0.5% procline- |
| | 300 |
| Wash buffer | 50 mM pH 7.4 Tris + 0.1% Tween-20 + 0.5% BSA + 0.5% |
| | procline-300 |
| Magnetic bead | 0.02M pH 7.4 PB + 0.1% BSA + 0.5% procline-300 (Seven |
| working | fluorescent magnetic beads with different green fluorescence |
| solution | intensities) |
| Antibodies, etc. | Capture antibodies for IL-2, IL-5, IL-7, IL-8, IL-9, IL-12, IL- |
| | 15, IL-2, IL-5, IL-7, IL-8, IL-9, IL-12, IL-15 biotinylated |
| | detection antibodies, streptavidin HRP, IL-2, IL-5, IL-7, IL- |
| | 8, IL-9, IL-12, IL-15 antigens |

### 2. Coating Method of Magnetic Beads

The magnetic bead coating method of this example was EDC coupling method, and the specific operation steps were as follows:
(1) The self-made carboxyl magnetic beads with different fluorescence intensities were mixed thoroughly in advance.
(2) Washing: an Ep tube was taken and 20 uL of 100 mg/mL carboxyl (2 mg) magnetic beads were added, and 1.8 mL of 100 mM MES at pH 5.0 was added. The tube was inverted and washed for 3 minutes, placed on a magnetic frame for 3 minutes. The supernatant was removed. This washing step was repeated twice.
(3) Activation:

Activation solution 1: 100 mg/mL EDC was prepared: 10 mg EDC was weighed and added with 100 uL pre cool 100 mM MES at pH 5.0, dissolved.

Activation solution 2: 100 mg/mL NHS was prepared: 20 mg NHS was weighed and added with 200 uL pre cool 100 mM MES at pH 5.0, dissolved.

The amount was prepared according to demand.
(4) The liquid was quickly added to the prepared magnetic beads in the order shown in Table 8, rotated at room temperature for 60 minutes, added in a ratio of 1:1:2.

TABLE 8

| | | |
|---|---|---|
| 100 mM pH 5.0 pre cooled MES | 5 | uL |
| Mix 100 mg/mL EDC (first) mixed well and then | 5 | uL |
| added NHS | | |
| 100 mg/mL NHS | 50 | uL |

(5) Washing: the mixture was placed on a magnetic frame to adsorb for 4 minutes to remove the supernatant, added with 1.8 mL of ice cold 100 mM ice MES pH 5.0, placed on a magnetic frame for 3 minutes, and the supernatant was removed. This washing step was repeated 4 times.
(6) Binding antibodies:

For example, magnetic beads: antibody=50:1 for coupling, with a total volume of no less than 250 uL as shown in Table 9. The concentrations of capture antibodies for IL-2, IL-5, IL-7, IL-8, IL-9, IL-12, and IL-15 were calculated according to the instructions provided by the respective manufacturers, and preservatives, glycerol, and other components were removed by dialysis. The mixture was mixed well and combined at room temperature for 60 minutes.

TABLE 9

| Ratio of magnetic bead to antibody | 50:1 | |
|---|---|---|
| Magnetic beads | 2 | mg |
| Antibody weight | 0.04 | mg |
| Antibody volume | (Added according to antibody | |
| | concentration) | |
| 0.02M pH 8.0 boric acid buffer solution | (Added according to antibody | |
| | volume) | |
| Total volume | 250 | uL |

(7) Blocking: 1.8 mL of 50 mM pH 7.4 Tris+0.1% Tween-20+1% BSA buffer were added and washed 3 times. 1.8 mL of 50 mM pH 7.4 Tris+0.1% Tween-20+1% BSA 0.5% Proclin buffer were added again and incubated at room temperature for 90 minutes.
(8) Washing: the mixture was placed on a magnetic frame for 3 minutes, the supernatant was removed, 1.8 mL of 0.02M pH 7.3 PB+0.1% BSA+0.5% ProcLin buffer were added, and placed on a magnetic frame for 3 minutes. This washing step was repeated 3 times.
(9) Preparation of working solution: 16.67 mL of 0.02M pH 7.3 PB+0.1% BSA+0.5% Proclin buffer were added to 120 ug/mL magnetic beads and stored at 2-8° C.
(10) the solution was prepared to a concentration of 10 million magnetic beads/mL, and the above IL-2, IL-5, IL-7, IL-8, IL-9, IL-12, IL-15 and other magnetic bead solutions were mixed before use.

### 3. Detecting Method

(1) 50 uL of a series of samples of IL-2, IL-5, IL-7, IL-8, IL-9, IL-12, IL-15 solutions were taken, including the configuration of different concentrations of calibrators, for a mixture of 5 concentrations including 0, 5, 10, 100, 500fg/mL.
(2) 50 uL of magnetic beads was mixed with 50 uL of sample, reacted at 37° C. for 15 minutes. Afterwards, the magnetic beads were washed three times with 200 uL of PBST using a magnetic frame.
(3) 100 uL biotin coated detection antibody was added and reacted at 37° C. for 15 minutes. Afterwards, the magnetic beads were washed three times with 200 uL of PBST using a magnetic frame.
(4) 100 uL of HRP labeled with streptavidin (1:5000 fold dilution) was added and reacted at 37° C. for 15 minutes. Afterwards, the magnetic beads were washed three times with 200 uL of PBST using a magnetic frame.
(5) After the above reaction was completed, the magnetic beads were dispersed into 50 μL of PBS solution.

### 4. Droplet Generation

(1) 50 uL of pre cooled ADHP fluorescence generating substrate was added immediately, mixed well, and oil for droplet generation 200 uL was added immediately.
(2) The head of the high-speed homogenizer was inserted into the above mixture, the speed was adjusted to high-speed mode (10,000 RPM), and homogenized for 10s.
(3) The above water-in-oil mixture was placed at 37° C. to react for 5 minutes.

### 5. Detection

(1) The above reaction solution was placed on a magnetic frame, and it was attractively washed three times with oil for droplet generation, and finally it was resuspended in 20 uL of oil for droplet generation.
(2) The above-mentioned magnetic bead droplet mixture was added to the detection chip to form a single-layer distribution, and it was placed under a microscope for observation and detection.
(3) Photos of the green and red channels were taken.
(4) The fluorescence images under two excitation conditions: excitation at 570 nm and emission at 585 nm, and excitation at 470 nm and emission at 490 nm were analyzed. And the number of fluorescent droplets or magnetic beads under the two channels was counted. Droplets that did not contain magnetic beads were blank droplets with no fluorescence.
(5) The number of positive droplets containing magnetic beads and negative droplets containing magnetic beads were counted. The corresponding detection data list was shown in Table 10 below:

TABLE 10
Digital Immunoassay Data for IL-2, IL-5,
IL-7, IL-8, IL-9, IL-12, IL-15

| | Percentage of positive | Number of positive |
|---|---|---|
| | magnetic beads | magnetic beads |
| Concentration of IL2 | | |
| calibrator fg/mL | | |
| 0 | 0.0115% | 0 |
| 5 | 0.2119% | 943 |
| 10 | 0.3739% | 1534 |
| 100 | 3.2445% | 12718 |
| 500 | 8.1478% | 35211 |
| Concentration of IL5 | | |
| calibrator fg/mL | | |
| 0 | 0.0124% | 58 |
| 5 | 0.1382% | 633 |
| 10 | 0.5432% | 2375 |
| 100 | 4.6811% | 19457 |
| 500 | 6.0916% | 24572 |
| Concentration of IL7 | | |
| calibrator fg/mL | | |
| 0 | 0.0036% | 15 |
| 5 | 0.3248% | 1347 |
| 10 | 0.4082% | 1677 |
| 100 | 3.6324% | 16794 |
| 500 | 7.2676% | 31997 |
| Concentration of IL8 | | |
| calibrator fg/mL | | |
| 0 | 0.0022% | 10 |
| 5 | 0.3263% | 1597 |
| 10 | 0.6011% | 2706 |
| 100 | 4.1492% | 18274 |
| 500 | 7.0051% | 31682 |
| Concentration of IL9 | | |
| calibrator fg/mL | | |
| 0 | 0.0069% | 30 |
| 5 | 0.2982% | 1204 |
| 10 | 0.6187% | 2878 |
| 100 | 4.6866% | 18298 |
| 500 | 6.6575% | 25082 |
| Concentration of IL12 | | |
| calibrator fg/mL | | |
| 0 | 0.0105% | 44 |
| 5 | 0.2087% | 938 |
| 10 | 0.4562% | 1978 |
| 100 | 4.0261% | 17485 |
| 500 | 6.7996% | 30558 |
| Concentration of IL15 | | |
| calibrator fg/mL | | |
| 0 | 0.0022% | 10 |
| 5 | 0.3263% | 1452 |
| 10 | 0.6011% | 2897 |
| 100 | 4.1492% | 19354 |
| 500 | 7.0051% | 32330 |

Table 10 shows that effective quantitative analysis of multiple cytokines (IL-2, IL-5, IL-7, IL-8, IL-9, IL-12, IL-15) can be performed using seven fluorescent magnetic beads with different green fluorescence intensity, with a detection range of 0-500 fg/mL.

### Example 3

### Rapid Analysis of SARS-CoV-2 Antigen

### 1. Experimental Materials

The experimental materials for this example are shown in Table 11.

TABLE 11

| Reagent name | Reagent components |
|---|---|
| Magnetic bead | 20 mM PBS |
| washing solution | |
| CB buffer solution | 50 mM CB buffer solution (pH 9.5) |
| 3M ammonium | 3M ammonium sulfate solution (pH 9.5) dissolved in 1xCB |
| sulfate | |
| Wash solution | 20 mM PB PH 7.4 + 0.1% BSA + 0.9% Nacl + 0.05% Proclin300 |
| before magnetic | |
| bead blocking | |
| Blocking buffer | 50 mM Tris-HCl PH 7.4 + 0.5% BSA + 0.9% Nacl + |
| | 0.05% Proclin300 |
| Magnetic bead | 50 mM Tris HCl pH 7.4 + 0.5% BSA + 1.0% gelatin + 0.9% NaCl + |
| working solution | 0.05% Proclin300 |
| Magnetic beads | 3 um Tosylmactivated green fluorescent magnetic beads |
| Raw material | capture antibody for SARS-CoV-2 (antibody can recognize |
| | SARS-CoV-2 nucleocapsid protein), HRP labeled detection |
| | antibody (antibody can recognize SARS-CoV-2 nucleocapsid |
| | protein), recombinant SARS-CoV-2 antigen |

### 2. Coating Method

(1) A 2 mL centrifuge tube was taken, 30 uL (3 mg) of Tosylactivated green fluorescent magnetic beads were added, and the magnetic beads were washed 3 times with 0.3 mL of 20 mM PBS at a speed of 30 times/minute, and adsorbed for 5 minutes each time. After washing, the washing solution was absorbed on a magnetic frame.
(2) The required raw materials, CB buffer solution, and 3M ammonium sulfate were added in proportion, as shown in Table 12.

TABLE 12

| | | |
|---|---|---|
| Ingredients/Magnetic Beads | 1 | mg |
| Coating antibody raw | 200 | uL |
| materials + CB buffer solution | | |
| 3M ammonium sulfate | 100 | uL |
| Total volume | 300 | uL |

Note: The final concentration of ammonium sulfate in the coating solution is 1M/L, and the minimum coating volume should not be less than 300 uL.
(3) The centrifuge tube in step 2 was placed on a multi-purpose rotating shaker and reacted at 2-8° C. for 20 hours.
(4) The centrifuge tube in step 3 was placed on a magnetic frame for 5 minutes, the supernatant was removed, 0.3 mL of wash buffer before magnetic bead blocking was added, and washed three times.
(5) 0.3 mL of blocking buffer was added to the washed magnetic beads from step 4, mixed well, and reacted on a multi-purpose rotary shaker at 37° C. for 6 hours.
(6) step 4 was repeated.
(7) The magnetic beads were resuspended, 0.3 mL of magnetic bead working solution was added to each tube, and it was placed on a multi-purpose rotating shaker at 37° C. for 24 hours, with a rotation speed of 30 times/minute.
(8) The centrifuge tube in step 3 was placed on a magnetic frame for 5 minutes, the supernatant was removed, 0.3 mL of wash buffer before magnetic bead blocking was added, washed three times for 5 minutes each time, and adsorbed on a magnetic frame for 5 minutes to remove the washing solution.
(9) The washed magnetic beads were added to the magnetic bead working solution to prepare a 120 ug/mL magnetic bead working solution, and it was stored at 2-8° C.
(10) The solution concentration was configured to 10 million magnetic beads/mL.

### 3. Detecting Method

(1) A series of 50 uL antigen standard solutions were taken, including 0, 50, 100, 100, 1,000, 5,000, 10,000 fg/mL.
(2) 50 μL of magnetic beads was mixed with 50 uL of sample and reacted at 37° C. for 5 minutes. Afterwards, the magnetic beads were washed three times with 200 uL of PBST using a magnetic frame.
(3) 100 uL HRP labeled detection antibody (0.1 ug) was added and reacted at 37° C. for 5 minutes. Afterwards, the magnetic beads were washed three times with 200 uL of PBST using a magnetic frame.
(4) After the above reaction was completed, the magnetic beads were dispersed into 50 uL of PBS solution.

### 4. Droplet Generation

(1) 50 uL of pre cooled ADHP fluorescence generating substrate was added immediately, mixed well, and oil for droplet generation 200 uL was added immediately.
(2) The head of the high-speed homogenizer was inserted into the above mixture, the speed was adjusted to high-speed mode (10,000 RPM), and homogenized for 10s.
(3) The above water-in-oil mixture was placed at 37° C. to react for 5 minutes.

### 5. Detection

(1) The above reaction solution was placed on a magnetic frame, and it was attractively washed three times with oil for droplet generation, and finally it was resuspended in 20 uL of oil for droplet generation.
(2) The above-mentioned magnetic bead droplet mixture was added to the detection chip to form a single-layer distribution, and it was placed under a microscope for observation and detection.
(3) Photos of the green and red channels were taken.

### 6. Analysis

The fluorescence images under two excitation conditions: excitation at 570 nm and emission at 585 nm, and excitation at 470 nm and emission at 490 nm were analyzed. And the number of fluorescent droplets or magnetic beads under the two channels was counted. Droplets that did not contain magnetic beads were blank droplets with no fluorescence.

TABLE 13
Results of Rapid Analysis of SARS-CoV-2 Antigen

| | Percentage of | |
|---|---|---|
| Concentration of | positive magnetic | Number of positive |
| calibrator fg/mL | beads | magnetic beads |
| 0 | 0.3313% | 1424 |
| 50 | 0.5217% | 2463 |
| 100 | 1.0888% | 4367 |
| 500 | 3.0510% | 14256 |
| 5000 | 12.8219% | 53562 |
| 10000 | 14.0522% | 63423 |

Table 13 shows that using the method of this example to detect SARS-CoV-2 antigen standard can quantitatively detect the antigen in the range of 0-10000 fg/mL.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A digital immunoassay detection and analysis method for precise quantification of proteins, comprising the steps of:
(s1) providing an aqueous phase mixture comprising the following complexes:
(A) a positive signal complex: a magnetic bead—antibody—antigen—detection antibody—enzyme label, and/or a magnetic bead—antibody—antigen—detection antibody complex; and
(B) a negative signal complex: a magnetic bead—antibody complex;
(s2) mixing the aqueous phase mixture with a chromogenic substrate and an oil phase to form a first mixture;
(s3) mechanically shearing of the first mixture to form a sheared water-in-oil second mixture;
wherein, the second mixture comprises a first micro droplet and a second micro droplet; the first micro droplet is a micro droplet containing a positive signal complex; the second micro droplet is a micro droplet containing a negative signal complex; and
(s4) measuring the first micro droplet to obtain the number of the first micro droplets and obtain the quantitative detection result of the proteins.

2. The method according to claim 1, further comprising the following sub steps in step (s1):
(s1a) providing an aqueous monodisperse magnetic bead solution;
(s1b) adding a certain proportion of capture antibodies to the magnetic bead solution to couple the magnetic beads with the capture antibodies, thereby forming a magnetic bead—antibody complex, followed by washing;
(s1c) adding an antigen to the solution containing the magnetic bead—antibody complex and reacting to form a magnetic bead—antibody—antigen complex, followed by washing;
(s1d) adding a detection antibody containing biotin or horseradish peroxidase (HRP) to a solution containing the magnetic bead—antibody—antigen complex and reacting to form a magnetic bead—antibody—antigen—detection antibody complex, followed by washing;
wherein, when a detection antibody containing biotin is added, proceed to step (s1e); when a detection antibody containing horseradish peroxidase is added, proceed directly to step (s2);
(s1e) adding an enzyme labeled molecule to a solution containing the magnetic bead—antibody—antigen—detection antibody complex to form a magnetic bead—antibody—antigen—detection antibody—enzyme label, followed by washing; and
(s1f) optionally, adding a negative signal antibody against the capture antibody to a solution containing the magnetic bead—antibody—antigen—detection antibody—enzyme label, so that the magnetic bead is labeled with a negative signal, followed by washing.

3. The method according to claim 1, wherein the mechanically shearing is carried out by homogenizing the first mixture using a high-speed homogenizer at a speed ranging from 2,000 to 100,000 RPM.

4. The method according to claim 1, wherein the total content of the first and second micro droplets in the water-in-oil second mixture is less than 30%, preferably less than 20%, and more preferably less than 10%, relative to a total content of empty micro droplets in the second mixture.

5. The method according to claim 1, further comprising the following steps between steps (s3) and (s4): washing the sheared water-in-oil second mixture to remove empty droplets that do not contain any first or second micro droplets.

6. The method according to claim 5, wherein the washing is carried out by adsorbing and intercepting micro droplets wrapped with individual magnetic beads through magnetic force, thereby washing and enriching the magnetic beads.

7. The method according to claim 1, wherein the magnetic bead is a magnetic bead with fluorescent properties or a magnetic bead without fluorescent properties.

8. The method according to claim 1, wherein in step (s3), the ratio D1/D2 of a diameter D1 of the first micro droplet to a diameter D2 of the magnetic beads contained therein is 1.2-20, preferably 1.5-5, more preferably 1.6-4, and more preferably 1.8-3.

9. The method according to claim 1, wherein the method is used for measuring n proteins, and the value of n is 1-200, preferably 1-20, and more preferably 1-10.

10. A digital immunoassay device for quantification of proteins, wherein the device comprises:
(a) a mixing module, which is configured to form an aqueous phase mixture comprising the following components:
(A) a positive signal complex: a magnetic bead—antibody—antigen—detection antibody—enzyme label, and/or magnetic bead—antibody—antigen—detection antibody complex; and
(B) a negative signal complex: a magnetic bead—antibody complex;
(b) a shearing module, which is configured to add a chromogenic substrate and an oil phase to the aqueous phase mixture to form a first mixture; and perform a mechanical shearing to form a sheared water-in-oil second mixture, which contains first and second micro droplets; the first micro droplet is a micro droplet containing a positive signal complex; the second micro droplet is a micro droplet containing a negative signal complex; and
(c) a digital detection module, which is configured to measure the first micro droplet, thereby obtaining the number of the first micro droplet and obtaining the quantitative detection result of the proteins.
